(19) United States
(12) Patent Application Publication
(10) Pub. No.: US 2020/0224156 A1
(43) Pub. Date: Jul. 16, 2020

(54) METHOD FOR REMOVAL OF YOLK

(71) Applicant: CRYOGENETICS AS, HAMAR (NO)

(72) Inventors: LINE CECILIE GRONENG, HAMAR (NO); MARI KJOS, FURNES (NO)

(21) Appl. No.: 15/758,839

(22) PCT Filed: Sep. 9, 2016
(86) PCT No.: PCT/EP2016/071318

§ 371 Date: Mar. 9, 2018

Related U.S. Application Data
(60) Provisional application No. 62/215,767, filed on Sep. 9, 2015.

(51) Int. Cl. C12N 5/073

(52) U.S. Cl. CPC C12N 5/0605; C12N 2506/02

(57) ABSTRACT

A method for isolating the blastoderm of a non-human vertebrate embryo by enzymatic removal of the yolk.

### FIELD OF THE INVENTION

The present invention relates to a method for yolk removal from embryos and isolation of blastoderms, in particular from fertilized embryos of aquatic origin.

### BACKGROUND OF THE INVENTION

Embryogenesis is the process where an embryo develops from the fertilization of the ovum. In normal development, the newly fertilized egg (zygote) undergoes a rapid series of mitotic divisions resulting in a larger and larger number of smaller and smaller cells producing a cluster of cells. This first period of division is not accompanied by an increase in the overall size of the embryo, but resulting in tightly packed mass of cells (blastomeres) forming a blastoderm. As the cleavage continues, the embryo enters the blastula stage, and the blastula cells begin to migrate into the embryo, marking the beginning of the next stage, the gastrula stage, where the gastrulation process begins.

All vertebrate embryos undergo a similar pattern of development after fertilization, the initial cleavage, blastulation, gastrulation and the phylotypic stage at which they all more or less resemble each other and show the specific features of notochord, somites and neural tube.

The pattern of division is influenced by the presence and location of yolk, which varies in the various vertebrates, some being more yolk rich than others. For example, upon division and formation of the cluster of blastomeres, frog embryo and the embryos of some fish species results in a very asymmetrical blastula, where the yolk of the embryo is concentrated towards the lower end of the embryo (vegetal pole), and the blastomeres is concentrated in the opposite end (animal pole).

Embryos of reptiles and birds, and some fish species, are composed almost entirely of yolk, where division of the cells occurs only in a tiny disc in one end of the embryo, named the blastodisc, resulting in an embryo that are not spherical.

Embryos having an uneven distribution of yolk in the cytoplasm of the ovum are found in embryo of birds, reptiles, and fish, and undergoes discoidal meroblastic cleavage, i.e. where the yolk is not incorporated in the cells during division.

The embryos of mammals has several similarities with reptilian eggs, except that they contain very little yolk, and the mammalian egg is holoblastic, forming a ball of cells surrounding a blastocoel, with an inner cell mass concentrated at one pole.

This inner cell mass is analogue to the blastodisc of reptiles, birds and fish like zebrafish.

The removal of yolk from a vertebrate embryo, such as fertilized embryos in the blastula stage, may be useful for a number of reasons. For example, the removal of egg yolk may be necessary in order to perform proteomic experiments on early stage non-human embryos, such as zebrafish embryos (see e.g. Link et al., BMC Developmental Biology, 2006, 6:1 (http://www.biomedcentral.com/1471-213X/6/1).

The removal of yolk of an embryo may furthermore be useful prior to isolation of genome sequences in order to perform further genetic analysis, e.g. to avoid and remove components in the egg yolk (proteins etc.) that may interfere with subsequent DNA isolation procedures and genetic analysis/gene sequencing. The removal of yolk from an embryo may also be useful in cell and tissue transplantation procedures and studies, as well as stem cell research. Furthermore, the removal of yolk from an embryo may be useful prior to cryoconservation of an embryo and for the development of embryo cryoconservation methods.

Cryoconservation of embryos in order to preserve the genome of endangered species or genetically valuable animals for farming or research purposes has become more important in the recent years.

Successful preservation of whole fish embryos has so fare shown to be difficult (cf. Ninhaus-Silveira et al. (2009), Zygote, 17(1), 45-55), and as an alternative to cryoconservation of whole fish embryo, cryoconservation of blastoderms has been suggested. Inter alia, Babiak et al., 2008, International Journal of ichthyology, 32(2) suppl., 139-141, reports about the cryoconservation of rainbow trout blastoderms, where the blastoderms at the mid late blastula stage were isolated after microsurgical cutting a chorion prior to cryoconservation. Furthermore, Kusuda et al. (2002), in Cryobiology, 45, 60-67 reports of the cryoconservation of dissociated chum salmon blastomeres. Prior to cryoconservation, the embryos are manually dechorionated and the dissociated blastomers are manually collected by dissection from the yolk cells.

The Zebrafish (Danio rerio), originally found in tropical freshwater in Himalayan region, has become widely used as a model organism for a several application in research as it possesses a number of advantages compared with other model system, in particular model systems involving higher vertebrates. Inter alia, zebrafish has been widely used in studies of vertebrate embryogenesis, organ development, and genetics both by independent research institution and the pharmaceutical industry. The use of zebrafish as a model organism has resulted in advances in the field of developmental biology, genetics, neurobiology, oncology, genetics, reproductive studies, stem cell research, teratology, toxicology, etc.

The removal of yolk from zebrafish for conservation of genetic material (gene banking), including conservation of zebrafish embryo, is desired in order to ensure proper storage and preservation of the genetic material of numerous individual zebrafish of importance obtained e.g. through such scientific and medicinal research. Methods for removal of yolk from zebrafish embryos would in addition allow for further studies on embryonic development.

Mizuno et al., 1999, Methods Mol Biol, 127:15-28 describes transplantation techniques, involving inter alia the removal of blastoderm cells from the yolk mechanically with a glass needle.

Kostomarova (1969), J. Embryol. Exp. Morph, 22:3, 407-430 isolated blastoderm of Misgurnus fossils L. by first incubating fertilized eggs with a trypsin solution in order to remove the egg shells, and thereafter by subjecting the trypsin treated eggs to centrifugation.

In Rivera et al., 2007, Developmental Dynamics, 236:489-493, a method is described where enzymatic digestion and mechanical manipulation is utilized in order to remove the parietal yolk sac of mouse embryos. In particular, a method is disclosed involving the use of a mixture of three enzymes, i.e. collagenase type IV, hyaluronidase and dispase, in combination with pipetting, to remove the parietal yolk sac. Whereas collagenase type IV and dispase is proteases cleaving specific proteins and/or polypeptides, hyaluronidase is an enzyme that degrade hyaluronic acid.

Collagenase type IV is a protease that cleaves the bond between a neutral amino acid (X) and glycine in the sequence Pro-X-Gly-Pro, a sequence that are found in high frequency in collagen. Collagenase type IV is isolated from Clostridium histolyticum. Dispase, present in Bacillus polymyxa, is a protease that cleaves fibronectin, collagen IV and to a lesser extent collagen. Hyaluronidase is an enzyme that degrades hyaluronic acid, an anionic, non-sulfated glucosaminoglycan.

However, the method described by Rivera et al. is time consuming and few embryos are treated at the same time. It also requires use of a pipette to isolate the blastoderm mechanically.

Russian patent No. 932399, published the 30 May 1982 relates to a method of inhibiting ribosomal RNA synthesis by incubating biological material in a sodium selenite solution. In SU932399, ten hour stage of embryonal development of inseminated embryos originating from female loaches are washed with 200 ml 0.5% trypsin for 7 minutes to destroy their embryonic membranes. A ten hour stage of embryonal development indicate that the embryos washed in trypsin was in the post blastula stage/early gastrula stage.

There is still a need for alternative and more efficient processes for isolating blastoderms from embryos by removing egg yolk.

### SUMMARY OF THE INVENTION

The present invention provides a method for the removal of egg yolk from an embryo by enzymatic digestion, in particular a non-human embryo of aquatic origin, using one or more proteases, in particular one or more non-specific protease.

The present invention provides the use of at least one protease for the removal of embryonic yolk of one or more non-human vertebrate embryos, provided that said mixture does not comprise collagenase and dispase.

More particularly, the present invention provides at least one protease for the isolation of one or more blastoderm of aquatic origin by the removal of embryonic yolk from said embryos, wherein the non-human embryo is a telolecithal embryo.

According to one embodiment, the mixture comprises one or more non-specific proteases(s), such as pronase.

The present invention furthermore provides a method for the removal of the yolk of one or more vertebrate embryos of aquatic origin comprising treating the one or more embryos with at least one protease for a period of time sufficient for the yolk to loosen from the one or more blastoderm.

In particular, a method is provided for isolation of one or more non-vertebrate blastoderms comprising the steps of:
a) removing the yolk of one or more vertebrate embryos of aquatic origin comprising treating the one or more embryos with at least one protease for a period of time sufficient for the yolk to loosen from the one or more blastoderm, and
b) inactivating or removing the protease;
c) collecting the resulting one or more blastoderms;
wherein the one or more embryos is telolecithal embryos.

In particular, the present invention provides for the removal of yolk of one or more vertebrate embryo of blastula stage.

In particular, the present invention provides for the removal of yolk of one or more embryos of 512-1K cell stage.

In particular, the present invention provides for the removal of yolk of one or more embryos of 21 Somite cell stage.

According to one embodiment, said mixture comprises one or more non-specific protease, such as pronase.

According to one embodiment of the above method, said method comprises the steps of
a. providing one or more vertebrate embryos or aquatic origin;
b. treating the one or more embryos with a non-specific protease for a period of time sufficient for the yolk to loosen from the remaining embryo, such as from one or more blastoderm;
c. inactivating or removing the said protease;
d. washing the one or more blastoderms in a physiologically acceptable salt solution.

According to one embodiment the embryos treated according to the present method is in the blastula stage, and the embryo is treated with the non-specific protease for a period of time sufficient for the yolk to loosed from one or more blastoderm

According to yet another embodiment, the embryo to be treated according to the present invention is in the blastula stage. According to one embodiment, the embryos are of the 512-1K cell stage. According to one embodiment, the embryos are of the 18 Somite cell stage. According to yet another embodiment, the embryos to be treated according to the present invention is zebrafish embryos of the 512-1K cell stage. The embryos may furthermore according to one embodiment be treated with the protease for at least 15 minutes, such as for at least 20 minutes, such as for at least 30 minutes.

According to one embodiment, the embryos of aquatic origin isolated according to the present invention is exposed for a protease, such as a non-specific protease, for about 30 minutes.

The present invention furthermore provides a solution comprising a blastoderm of aquatic origin essentially free of yolk. According to one embodiment, the present invention provides a solution comprising i) a blastoderm of aquatic origin essentially free of yolk and ii) at least one protease that do not originates from the blastoderm.

### FIGURES

FIG. 1 is a schematic illustration of an embryo where the yolk is concentrated towards the lower end of the egg, forming a vegetal pole, and where the other end, the animal pole, comprising rapidly dividing cells (FIG. 16-2, Molecular Bilogoy of the Cell, 2nd Ed., Alberts et al., 1989, Gerland Publishing Inc., New York & London, ISBN 0-8240-3695-6).

FIG. 2 shows the development of an zebra fish embryo, from the very early stage of cell division, to the about the middle of the blastula stage as illustrated by Kimmel et al. (1995) in Developmental Dynamics, 203:253-310.

FIG. 3 show blastoderms that was isolated from the egg yolk of zebrafish embryos of 512-IK cell stages according to the present method. The round blastoderm in the middle is pictured immediately after the pronase treatment, whereas the kidney formed blastoderm is pictured 12-24 hr after the pronase treatment.

FIG. 4 shows an embryo of the 21 Somite stage, wherein the whole yolk sac have been removed by the method of the present invention by treatment of the embryo of the 21 Somite stage.

FIG. 5 shows embryos at the Prim16 stage, wherein the egg yolk was removed by the method of the invention of the embryos of the Prim 16 stage. The whole egg yolk was removed for the embryo to the right after contacting the embryo with 1 mg/ml pronase solution, pre-warmed at 25° C. for 15 minutes.

FIG. 6 shows an embryo at the 18 somite stage (18 hr) for which about the half of the egg yolk where removed by the method according to the present invention at 18 somite stage.

FIG. 7 is a picture of an intact donor embryo (left) and an isolated blastoderm (right) isolated according to the present invention.

FIG. 8 shows the isolated blastoderm shown in FIG. 7 after transplantation on a donor yolk short time after transplantation.

FIG. 9 shows the transplanted embryo shown in FIG. 8 1-2 hours after transplantation of the blastoderm to the donor yolk.

FIG. 10 shows a picture of an isolated blastoderm after removal of yolk from a fertilized Puntius conchonius embryo in 32 cell stage.

FIG. 11 shows a picture of an isolated blastoderm after removal of yolk from a fertilized Puntius conchonius embryo in 512 Cell stage.

FIG. 12 shows a picture of an isolated blastoderm after removal of yolk from a fertilized Puntius conchonius embryo in 1K cell stage.

FIG. 13 shows a picture of an isolated blastoderm after removal of yolk from a fertilized Puntius conchonius embryo in middle blastula period, stage High (past 1K cell stage).

### DETAILED DESCRIPTION OF THE INVENTION

The present invention is not limited to the particular methodology, protocols, or reagents described herein. The terminology used to describe particular embodiments is not intended to limit the scope of the present invention, which will be limited only by the appended claims. As used herein, the singular forms “a”, “and”, and “the” include plural reference unless the context clearly dictates otherwise.

Thus, for example, reference to “a blastoderm” is a reference to one or more blastoderms and includes equivalents thereof known to the skilled person.

Unless defined otherwise, all technical and scientific terms and expressions used herein have the same meaning as commonly understood to the skilled person to which this invention belongs, unless a particular definition is provided thus dictating otherwise.

### Definitions

The term “telolecithal embryo” as used herein refers to an embryo having an uneven distribution of yolk, i.e. where the yolk is concentrated at one pole of the embryo and being separate from the developing embryo.

For the purpose to the present invention, a telolecithal embryo is of aquatic origin, such as teleost embryos. Said teleost may be a fresh water fish or a marine fish. For example, the one or more non-human vertebrate embryo used according to the present invention may be one or more embryo from fish belonging to the family Cyprinidae and Clariidae. According to one embodiment, the embryos used according to the present invention are selected from the group consisting of carps, minnows, barbs, barbels. In particular, the method according to the present invention is applicable for the removal of yolk and isolation of blastoderms from fish selected from the group consisting of Danio rerio (zebrafish), Barbus sachsii and Puntius conchonius.

The present invention provides a new and alternative method for removal of yolk from embryos, such the yolk of embryos in the blastula stage. In particular, the present invention provides a method for removal of yolk and isolation of blastoderms.

An embryo is a multicellular diploid eukaryote in its earliest stage of development. The term «embryo» is understood to cover vertebrate embryos of a variety of species, and in particular vertebrate embryos of aquatic organisms.

During embryogenesis, the embryo develops through various stages, beginning with first cleavage stage prior to the forming of the blastula stage. The method according to the present invention is inter alia applicable for the removal of yolk from embryos that undergo meroblastic cleavage, i.e. embryos comprising large amounts of yolk, such as telolecithal embryos.

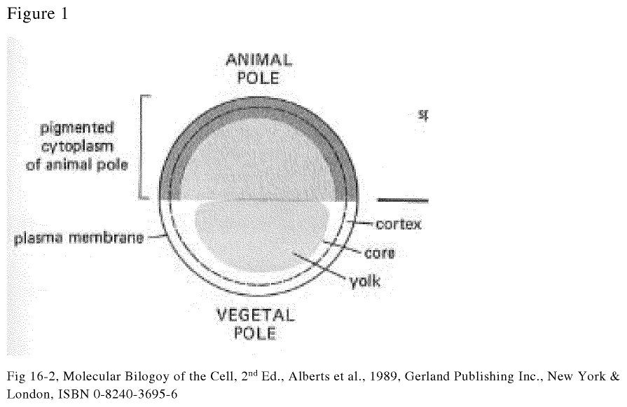

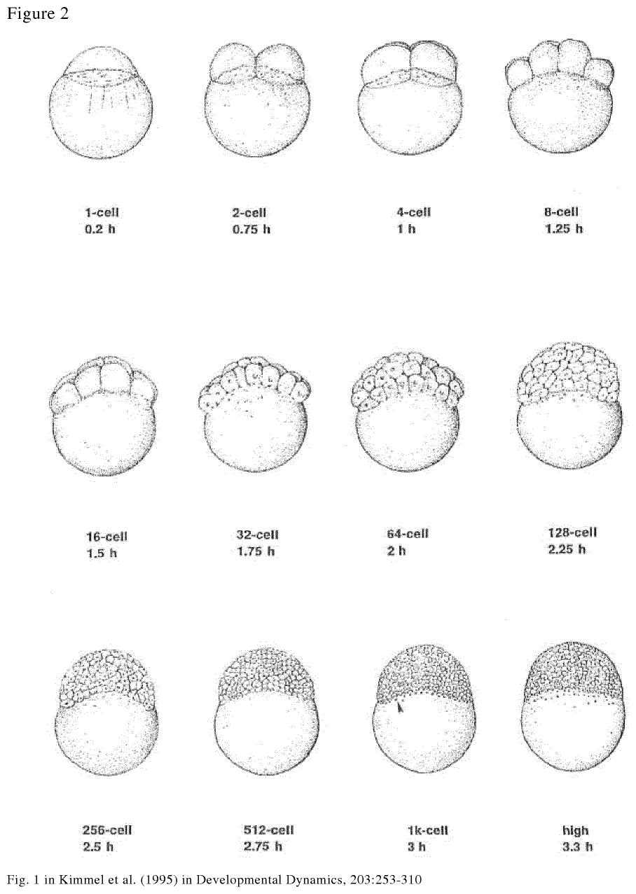

In particular, the present method is useful for removal of yolk from embryos that have undergone one of the major types of meroblastic cleavage, i.e. discoidal cleavage where cells formed by the cleavage process do not penetrate the yolk. The cleavage results in that most of the yolk is concentrated toward the lower end of the egg, named the vegetal pole whereas the cleaved cells is positions at the opposite end, named the animal pole (FIG. 1 and FIG. 2).

The second stage following the initial cleavage stage is the blastula stage, and the cells of the blastula stage, is called blastomeres. For e.g. zebrafish, the blastula stage typically starts at 128 cell stage, about 2 hours after fertilization of the egg, and up to about 4.5 hr after fertilization of the egg (Kimmel et al, supra). FIG. 2 illustrates the development of a fertilized zebrafish egg, from the very first cleavage up to about the middle of the blastula stage (>1K cells).

The term “blastoderm” is according to one embodiment to be understood to mean the layer of cells positions at one pole of fertilized eggs at the blastula stage, such as the blastula stage of a yolky fish embryo.

The next stage is the gastrula stage, where the lump of blastomers is transformed into a multilayer structure. The gastrula period for e.g. zebrafish is from about 5 hr to about 10 hr after fertilization (Kimmel et al., supra).

After the gastrula stage, the embryo enters a segmentation period, the somitogenesis, where the somitomeres are formed and the cells of the embryo is differentiated into various types of cells that finally represents the different organs of the fetus to be formed. The segmentation period, or the somite stage, starts about 10 hours after fertilization of a zebrafish (Kimmel et al., supra).

The time to enter the various stages of an embryo may vary depending on the species of the embryo in questions and the temperature of the surroundings. The skilled person will be able to identify the most suitable stage of a particular embryo to be treated according to the present invention based on his general knowledge and the teaching of the present invention, as well as depending upon the purpose of the yolk removal.

According to one embodiment, the one or more embryos to be treated according to the present invention is an embryo of an aquatic organism. According to yet another embodiment, the embryo to be treated according to the present invention is a fish embryo, in particular originating from species forming an embryo of telolecithal structure.

According to one embodiment of the present invention, embryos treated according to the present invention are collected from a fish belonging to the family salmonidiae, such as e.g. Atlantic salmon.

In particular, the present invention is useful in isolating blastoderm by removal of egg yolk of fish, in particular to fresh water fish belonging to the family Cyprinidae and Clariidae, such as zebra fish (Danio rerio).

The present invention provides the isolation of one or more blastoderm from one or more telolecithal embryos. The method according to the present invention provides for the isolation of a blastoderm that may e.g. be transferred to a donor yolk and further development of the transplanted embryo to provide offspring, such as finally hatching of a fish fry.

The blastoderm to be transferred to a donor yolk includes an intact blastoderm, i.e. a blastoderm comprising the amount of blastomeres necessary in order to develop to a fully developed offspring. It is to be understood that an intact blastoderm includes a blastoderm where the blastomeres are not separated from each other but linked to form an intact blastoderm. A donor yolk may preferably be an intact yolk of the same specie as the blastoderm to be transplanted. The donor yolk may be obtained by any method known to the skilled person, such as e.g manually using a scalpel or by any other means.

A description of the stages of embryonic development of the zebrafish is found in Kimmel et al., 1995, Developmental Dynamics, 203: 253-310. The naming of the various stages of development of a zebrafish embryo is meant to be in accordance with the definitions presented therein.

According to one embodiment, the stage of a zebrafish embryo used as starting material in the present method should not exceed the Epiboly stage. According to yet another embodiment, when treating a zebrafish embryo according to the present invention, the embryo should be within the 512-1K cell stage of the embryonic development, i.e. about 2 and 3 hours of embryonic development at 28.5° C. post conception (cf. Kimmel et al., supra).

The present method, when applied to zebra fish embryos for the purpose of removing yolk and isolating the blastoderm, it is advantage to use embryo treated at early stages of blastula stage. Treatment of zebrafish embryos at beginning epiboly or later stages seems to give varying results with few or none isolated blastoderms.

It should be noted that although the present invention includes examples of the treatment of fish embryos, it is to be understood that the present method may also be used in order to isolate the blastoderm and remove the yolk from embryos of other organisms, wherein the embryo is of telolecithal structure, e.g. for studying embryonic development, or for which species, the storage of genetic material for later development of offspring is of interest.

A fertilized fish embryo may be obtained by placing female and male fish in suitable compartment, such as a mating tank, in order to allow mating. The skilled person will acknowledge than various fish mating protocols is available for various fish species. For example, in order to obtain fertilized zebrafish embryos, female and male fish may be placed separated in a container over night, and brought together for mating as soon as possible after the light are turned on the next morning. Zebrafish typically lay eggs within 2 hours after the lights come on.

Eggs are collected in separate Petri dishes and washed in e.g. egg water (laboratory grade water, pH 7, comprising methylene blue in order to inhibit bacterial growth), to remove debris and sort out infertile eggs. The fertile embryos may be stored in the Petri dish prior to being subjected to the method according to the present invention.

The method according to the present invention may also be used in order to remove the yolk and isolate the blastoderm of fish embryos obtained by artificial fertilization. Female eggs may e.g. be collected by manually applying pressure on the ventral side below the pectoral fins towards the pelvic fins resulting in that eggs are expressed through the cloacal opening and transferred to a Petri dish.

Fertilization is then obtained by contacting the isolated eggs with sperm collected from male fish according to methods well known to the skilled person. Sperm may e.g. be obtained by gently squeeze both sides of the fish from posterior to the pectoral fin towards the anal fin. The sperm is stored in well known diluents prior to fertilization.

The stage of the embryo to be treated according to the present invention may vary dependent upon the purpose of the removal of yolk as well as the type of species of embryo. It is to be understood that the yolk of a vertebrate embryo may be removed from various embryonic stages.

For example, for cryoconservation and/or transplantation of the zebrafish blastoderm onto a zebrafish donor yolk, the embryo to be treated is preferably within the blastula stage, preferably within the 512-1K stage.

However, dependent on the purpose of the removal of yolk from the embryo and isolation of blastoderm, i.e. for genetic analysis or embryo developmental studies, the yolk may also be removed at any other suitable stages of the embryo. For example, the yolk may be removed at the blastula stage, which may provide for the isolation of blastoderms. For the purpose of genetic analysis, the removal of yolk and isolation of blastoderm may be performed at any stage of the blastula stage. An advantage of the present invention is that the method according to the present invention provides for the isolation of intact blastoderms, i.e. where the blastoderm cells are not dissociated.

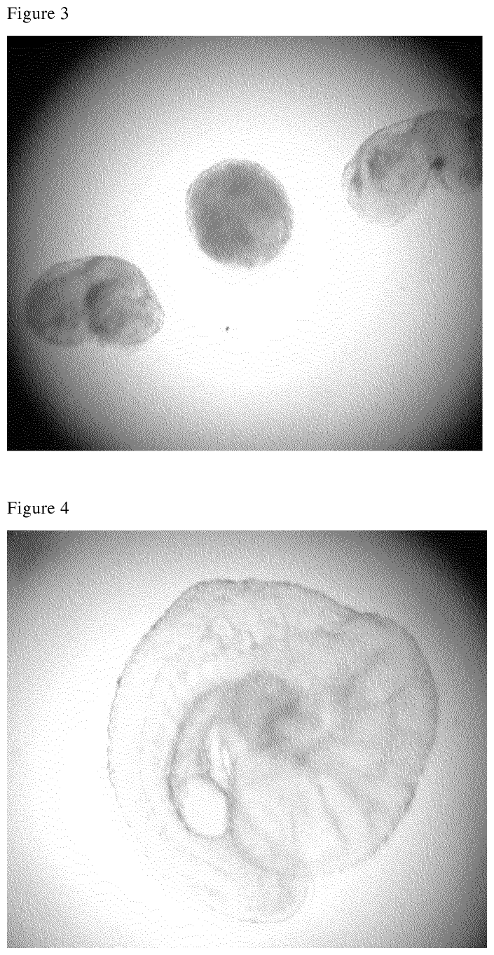

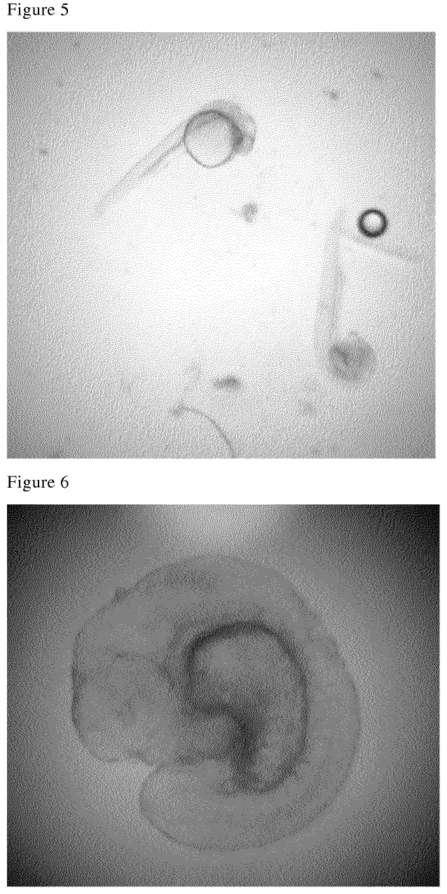

The yolk may also be removed at later embryonic stages according to the present invention, such as e.g. the somite stage or also later stages (e.g. about prim 15 stage, such as prim 16 stage). The present method have inter alia been used to remove the yolk of zebrafish embryos in the somite 18 stage, somite 21 stage and prim16 stage (cf. FIG. 4-6), as well as in the blastula stage (FIG. 3).

The term non-specific protease is to be understood to mean a protease or a mixture of protease that is non-specific in respect of substrate protein. A non-specific protease or a mixture thereof do not only degrade or cleave one specific type protein or a specific group of proteins but cleave various types of protein irrespective of the type of protein, its function and where and when in an organism a protein is expressed. In particular, it is to be understood that proteases that are not to be considered comprised by the definition of non-specific proteases given herein is e.g. Collagenase type IV and dispase.

A non-specific protease that is particularly useful according to the present invention is pronase. Pronase is commonly known as a mixture of proteases isolated from the extracellular fluid of Streptomyces griseus. Various manufacturers provide pronases that may be used to remove egg yolk according to the present invention. As a non-limiting example of a pronase useful according to the present invention is Pronase E (CAS 9036-06-0) provided e.g. by SERVA Electrophoresis, Heidelberg, Germany.

The concentration of the non-specific protease to be used according to the present invention may vary dependent upon the species of the embryo and the stages of development. The skilled person will, based on the teaching herein, be able to identify the preferable stages of the embryo dependent upon the species. According to one embodiment, the amount of pronase E is preferably within the area of 0.5 mg/ml-2 mg/ml depending on the stage of development when used to remove the egg yolk of one or more vertebrate embryos, in particular zebrafish embryos.

The amount and time of exposure of the embryo to the non-specific protease varies depending upon the type of non-specific protease used, the species and stage of the embryo development. The optimal time of exposure of the embryos with the non-specific protease may be found by observing the embryos during the treatment, and by transferring the embryos from the enzyme solution to the washing solution as soon as the embryos starts loosing the yolk sac. The embryos to be treated according to the present method should be contacted with the pronase in a sufficient time to allow the blastoderm to be released from the yolk. The skilled person will based on his general knowledge and based on the teaching of the present invention find the suitable amount and the suitable exposure time for the particular embryo to be treated.

For example, the skilled person may observe whether the isolated blastoderm is viable after the protease treatment and washing step of the present invention. For example, zebrafish blastoderms that are not viable after the steps of protease treatment and washing according to the present method will be grey or black of color, inflated with water. In case the isolated lump of blastomers dissociate, it indicates that protease should have been quenched/removed at an earlier time.

Various available proteases that may be used to remove yolk from an embryo according to the present invention are known to have an optimum temperature. Furthermore, embryos of different species may have different optimum temperatures. The skilled person will thus acknowledge a protease having an optimum temperature close to the optimum temperature of the embryo of questions may be an advantage when removing the yolk of an embryo according to the present invention.

After the protease treatment of the vertebrate embryo according to the present method, the protease may be quenched by any suitable method. The embryos may e.g. be washed in any suitable washing solution, such as e.g. a solution comprising Hank's Balanced Salt Solution (HBSS) and Fetal Bovine Serum (FBS). The embryos may be washed one or more times, for e.g. 5 minutes each time, for removal of the enzyme. According to one embodiment, zebra fish embryos exposed to a protease according to the present invention are preferably washed three times in order to stop the protease activity,

According to one embodiment of the present invention, embryos of the 512-1K cell stage is treated with 1 mg/ml pronase E pre-warmed at 25° C. for 30 minutes in order to remove the yolk sac. Said treatment results in that almost all embryos loose their yolk sac, or soon after the last washing step or within one hour after treatment.

The yolk sac removal treatment by pronase may be stopped by simply quenching and/or removing the enzyme from the container comprising the pronase treated embryos by washing.

In order to quench the protease activity, the protease may also be inactivated by any method well known to the skilled person, or in accordance with the methods and materials and recommendations provided by the various pronase manufacturers. For example adding a pronase inhibitor provided by various pronase manufactures.

When applying the present method on embryo of aquatic origin belonging to the family Cyprinidae and Clariidae, such as zebrafish embryos at stage 512-1K, the time of exposure is preferably within the area of 15-60 minutes, preferably within about 30 minutes when treated with about 1 mg/ml pronase, in particular pronase E.

The blastoderm isolated according to the present invention may also be used in proteomic analysis or genetic analysis.

The embryos remaining after the removal of yolk treated according to the present invention may be subjected to genetic or proteomic analysis. For example, genomic material may be isolated from the blastoderms or later stages of the embryo and further subjected to well known gene sequence techniques, such as e.g. PCR etc.

The embryos treated according to the present invention may also be subject to further developmental studies, such as being cultured in vitro for research purposes or for transplantation experiments.

The embryonic material remaining after the removal of yolk according to the present method, at any stage of the embryo, may be transferred to a suitable storage medium prior to subsequent handling as described above. The storage medium may depend upon the type of procedure to be performed.

According to one embodiment, blastoderms isolated by the present method may e.g. be further stored by cryoconservation (slow freezing or vitrification). Prior to cryoconservation, the blastoderms are treated with a suitable cryoprotectant, transferred to a suitable container suitable for cryoconservation of biological material, and cooled for long term storage in e.g. liquid nitrogen.

Upon thawing of the blastoderms, the blastoderms may thereafter be transplanted to a freshly isolated yolk, and cultured for further developing of the embryo.

### EXAMPLES

Materials used in yolk removal:
Pronase E (SERVA Electrophoresis GmbH)
Hanks' balanced salt solution, Cat no H8264 (SIGMA)
Hanks' balanced salt solution, Cat no H6648 (SIGMA)
FBS Superior, Cat. no. S 0615 (Biochrom)

### Example 1: Yolk Removal and Isolation of Blastoderms

Embryos used in this experiment are the results of natural breeding. Embryos were collected from fish kept in tanks at the zebrafish facility at the Norwegian Veterinary School, at a temperature of 27.5° C., light:dark cycle 14:10, and fed three times daily with artemia/SDS 400.

The pronase solution was pre-warmed to 25° C. and the further treatment is performed at RT. A number from −10 to −100 of embryos between the 512-cell to 1K cell stage was treated with 1 mg/ml pronase for 30 minutes in plastic petri dishes at room temperature. The treatment may also be performed at 25° C. in order for the removal of chorion and yolk to be performed more quickly.

The pronase treated embryos was then washed three times in Hank's Balanced Salt Solution (HBSS) with fetal bovine serum (FBS) to remove and quench the pronase. The isolated blastoderms are kept in HBSS comprising FBS until further use (e.g. transplantation, cryoconservation).

In FIG. 1, three blastoderms isolated according to the above protocol are pictured, and the results shows that the blastoderm soon after removal of the egg yolk, quickly attains a round shape (see the blastoderm in the middle, pictured immediately after pronase treatment and washing), whereas with time (12-24 hr), the blastoderm attains a more kidney-shaped form (see the blastoderm on the left and right side of FIG. 1).

Increasing the time of pronase treatment or pronase concentration seems not to have any beneficial effects on the isolation of blastoderms. The results furthermore show that the yolk is easier to remove when used to treat the embryo in the earlier stages. See FIG. 4-6.

### Example 2: Transplantation of Embryo Blastoderm on Donor Yolk

Zebrafish embryos was exposed to pronase and washed according to the method of example 1. The isolated blastoderm where then transplanted onto a donor yolk according to the method disclosed in Mizuno et al, From Methods in Molecular Biology, Vol 127, Cell and Tissue Transplantation in Zebrafish Embryos (1999) and Yamaha et al, (2001), Germ-line chimera by low-part blastoderm transplantation between diploid goldfish and triploid crucian carp.

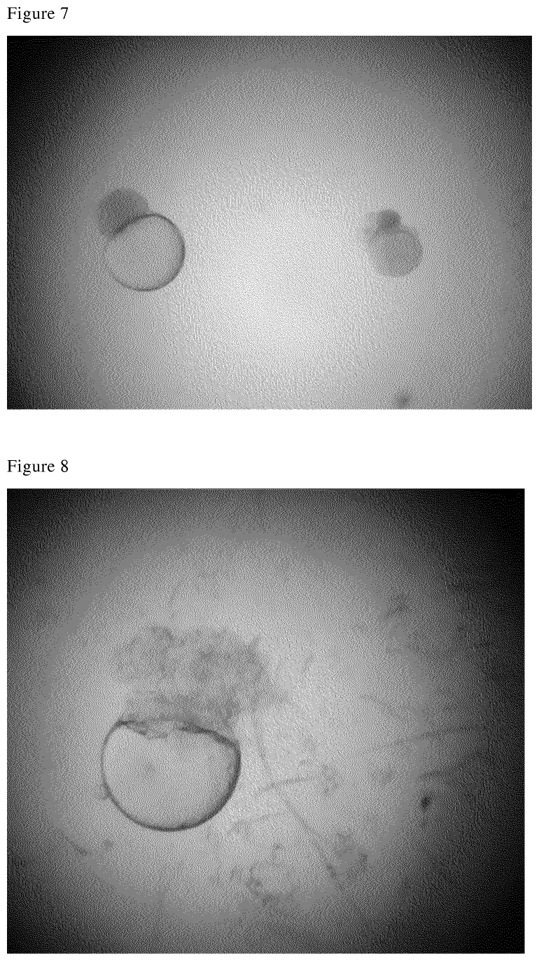

FIG. 7 is a picture of an intact donor embryo (left) and a blastoderm (right) isolated according to the present invention.

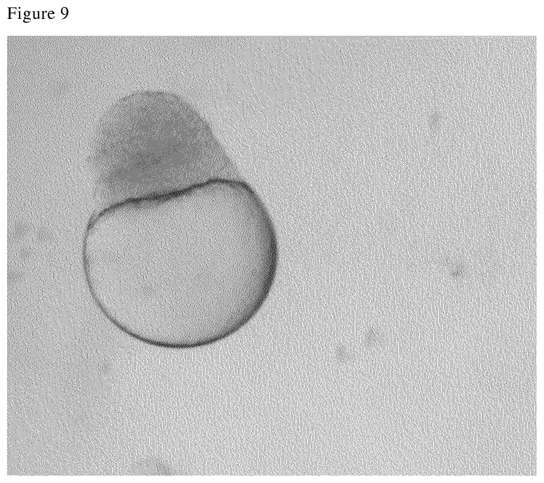

The blastoderm treated according to the present invention attached to the donor yolk short time after transplantation (FIG. 8), and developed normally thereafter, see FIG. 9 showing the transplanted embryo 1-2 hours after transplantation of the blastoderm to the donor yolk.

### Example 3: Yolk Removal and Isolation of Blastoderms from Carp

Embryos being the result of natural breeding of carp belonging to the species Puntius conchonius were treated in the same manner as disclosed in example 1.

As shown in FIG. 10-13, yolk were successfully removed resulting in isolation of blastoderms from Puntius conhonius embryos in the blastula stage.

The method of example 1 was also applied on naturally breeded embryos of goldfinned barbs (Puntius semifasciolatus) with similar results (data no shown).

1. Use of at least one protease for the isolation of one or more blastoderm from one or more vertebrate embryos of aquatic origin by the removal of embryonic yolk from said embryos, wherein the embryo is a telolecithal embryo.

2. Use according to any of the above claims, wherein the one or more proteases is non-specific proteases.

3. Use according to any of the above claims, wherein the non-specific protease is pronase.

4. Use according to any of the above claims wherein the vertebrate embryo of aquatic origin belonging to the order Cypriniformesis.

5. Use according to any of the above claims, wherein the vertebrate embryo of aquatic origin belonging to the family Cyprinidae and Clariidae.

6. Use according to any of the above claims wherein the vertebrate embryo of aquatic origin belonging to fish selected from the group consisting of carps, minnows, barbs, and barbels.

7. Use according to any of the above claims, wherein the vertebrate embryo of aquatic origin belonging to a specie selected from the group consisting of Danio rerio, Puntius conchonius and Puntius semifasciolatus.

8. Use according to any of the above claims, wherein the embryos is in the blastula stage.

9. Use according to any of the above claims, wherein the embryos is of 512-1K cell stage.

10. Use according to any of the above claims, wherein the embryos is of 21 Somite cell stage.

11. A method for isolation of one or more vertebrate blastoderms of aquatic origin comprising the steps of
d) removing the yolk of one or more embryos of aquatic origin comprising treating said one or more embryos with at least one protease for a period of time sufficient for the yolk to loosen from the one or more blastoderm, and
e) inactivating or removing the protease;
f) collecting the resulting one or more blastoderms;
wherein the one or more embryos of aquatic origin is a telolecithal embryo.

12. Method according to claim 12, wherein the mixture comprises one or more non-specific protease, such as pronase.

13. A method according to any of the claims 12-13, wherein the embryo is a fish embryo.

14. A method according to any of the claims 12-14, comprising the steps of
a. providing one or more embryos;
b. treating the one or more embryos with a non-specific protease for a period of time sufficient for the yolk to loosen from the remaining embryo, such as from one or more blastoderm;
c. inactivating or removing the said protease;
d. washing the one or more blastoderms in a physiologically acceptable salt solution.

15. A method according to any of the claims 12-15, wherein the embryo is in the blastula stage and the embryo is treated with the non-specific protease for a period of time sufficient for the yolk to loosed from one or more blastoderm.

16. A method according to any of the claims 12-16, wherein the said embryos is of 512-1K cell stage.

17. A method according to any of the claims 12-17, wherein the said embryos is of 21 Somite cell stage.

18. A method according to any of the claims 12-18, wherein the said embryos is treated with the protease for at least 15 minutes, such as for at least 20 minutes, such as for at least 30 minutes.

19. Solution comprising i) a blastoderm of aquatic origin essentially free of yolk and ii) at least one protease that do not originates from the blastoderm.
